(19) United States
(12) Patent Application Publication
(10) Pub. No.: US 2005/0038478 A1
(43) Pub. Date: Feb. 17, 2005

(54) Activation recovery interval for classification of cardiac beats in an implanted device

(76) Inventors: Ruth N. Klepfer, St. Louis Park, MN (US); John E. Burnes, Andover, MN (US)

Correspondence Address: MEDTRONIC, INC., 710 MEDTRONIC PARKWAY NE, MS-LC340, MINNEAPOLIS, MN, 55432-5604, US

(21) Appl. No.: 10/638,653

(22) Filed: Aug. 11, 2003

(51) Int. Cl. A61N 1/362

(52) U.S. Cl. 607/9

(57) ABSTRACT

The present invention provides a system and method for classifying cardiac beats based on activation-recovery intervals (ARIs) or an ARI-related parameter such as the spatial dispersion of activation, recovery or ARIs. The beat classification method may be used in monitoring and detecting cardiac rhythms and/or for controlling a cardiac stimulation therapy. The beat classification method includes acquiring a reference ARI for one or more known types of cardiac beats; measuring the activation-recovery interval of an unknown cardiac beat during cardiac activity monitoring; comparing the measured activation-recovery interval to the stored reference ARI(s); and classifying the cardiac beat based on the comparison between the measured ARI and the reference ARI(s).

### FIELD OF THE INVENTION

The present invention relates generally to the field of implantable cardiac stimulation devices and more specifically to a device and method for classifying cardiac beats based on measuring the activation-recovery interval and using this beat classification for controlling a cardiac stimulation therapy.

### BACKGROUND OF THE INVENTION

In the application of cardiac electrical stimulation therapies, it is often desirable to recognize the type of cardiac activity present in order to determine if a therapy should be delivered or withheld or if the therapy delivery parameters need adjustment. For example, in implantable cardioverter defibrillator devices, methods for detecting and discriminating tachycardia and fibrillation from normal sinus rhythm are used to determine when a cardioversion or defibrillation therapy is needed. In order to detect arrhythmias, a cardiac cycle, also referred to informally herein as a “beat,” that is a normal sinus beat must be distinguished from a pathological beat. During bradycardia pacing, cardiac resynchronization therapy, or other types of pacing therapies, it is important to distinguish an evoked depolarization in response to a pacing pulse from an intrinsic beat that occurs spontaneously following a pacing pulse if the pacing pulse is of insufficient energy to depolarize, or “capture”, the myocardium. Methods for classifying beats as “captured” beats or “loss of capture” beats are used in capture management methods developed to ensure pacing pulses are effective.

During some cardiac stimulation therapies, such as extra systolic stimulation for achieving post-extra systolic potentiation, it is undesirable to deliver a stimulation pulse when an abnormal beat occurs. A change in the activation pattern of the myocardium associated with an abnormal beat may result in a stimulation pulse being delivered during the vulnerable period of the cardiac cycle, which may induce an arrhythmia. Therefore, it is clear that in the field of cardiac stimulation therapies, reliable classification of cardiac beats is important for achieving safety and efficacy in a number of therapy applications.

Methods for classifying cardiac beats may rely on the detection of cardiac events observed on a cardiac electrogram (EGM), such as P-waves as evidence of atrial depolarization and R-waves as evidence of ventricular depolarization. The morphology of P-waves or R-waves and/or the intervals occurring between consecutive P-waves, R-waves or P- and R-waves may be used for detecting and classifying cardiac activity. One method for detecting and classifying cardiac rhythms is generally disclosed in U.S. Pat. No. 5,342, 402, issued to Olson et al., incorporated herein by reference in its entirety, which uses criteria for sensed events, event intervals, and event rates.

An arrhythmia detection and classification system that employs a prioritized set of inter-related rules for arrhythmia detection is generally disclosed in U.S. Pat. No. 5,545,186, issued to Olson et al., incorporated herein by reference in its entirety. The highest priority rule that is satisfied at a given time controls the behavior of the device in regard to the delivery or withholding of therapy. This methodology includes classification of sensed events into a limited number of event patterns. Certain sequences of event patterns are strongly indicative of specific types of heart rhythms.

An alternative approach to interval-based arrhythmia detection relies on EGM morphology analysis to discriminate a normal EGM morphology from an abnormal EGM morphology. U.S. Pat. No. 6,393,316, issued to Gillberg et al., incorporated herein by reference in its entirety, generally discloses a method and apparatus that uses a wavelet transform to discriminate normal and aberrantly conducted depolarizations. Wavelet transform analysis, as well as other morphology analysis methods, generally require greater processing time and power than interval-based detection methods. Accuracy of morphology-based detection algorithms alone may be limited due to myopotential noise, low amplitude EGM signals, waveform alignment error, and rate-dependent aberrancy. Therefore, wavelet transform analysis has been combined with detection interval criteria such that a wavelet transform is performed upon detection of a fast rate.

Some abnormal beats, however, may occur at intervals similar to normal sinus beats. Therefore, beat classification methods relying on interval and rate information for indicating an abnormal beat or when additional morphology analysis is needed can be “fooled” when intervals between sensed events indicate a normal beat when in fact the beat is an ectopic beat occurring near the sinus rate or the beat is occurring at a high rate that is not detected due to blanking intervals applied to sensing circuitry.

During cardiac pacing therapies, which may include bradycardia pacing, cardiac resynchronization therapy, extra systolic stimulation, anti-tachycardia pacing, overdrive pacing, or rate suppression pacing, it is important to deliver a pacing pulse of adequate energy to cause depolarization, or “capture”, of the myocardial cells. Many patents address methods for capture management, which often include detecting an evoked response following delivery of a stimulation pulse as evidence of capture. However, an intrinsic cardiac event or other non-cardiac signal may occur at approximately the same time that an evoked response is expected to occur. Therefore, detection of a cardiac event following a pacing pulse is not entirely specific in discriminating evoked responses from intrinsic responses or noise.

Extra systolic stimulation may be delivered to achieve the mechanical benefits of post-extra systolic potentiation (PESP). PESP is a property of cardiac myocytes that results in enhanced mechanical function of the heart on the beats following an extra systolic stimulus delivered early after either an intrinsic or pacing-induced systole. The magnitude of the enhanced mechanical function is strongly dependent on the timing of the extra systole relative to the preceding intrinsic or paced systole. When correctly timed, an extra systolic stimulation pulse causes an electrical depolarization of the heart but the attendant mechanical contraction is absent or substantially weakened. The contractility of the subsequent cardiac cycles, referred to as the post-extra systolic beats, is increased as described in detail in commonly assigned U.S. Pat. No. 5,213,098 issued to Bennett et al., incorporated herein by reference in its entirety.

One perceived risk of extra systolic stimulation, is that the extra systolic stimulation pulse, which must be carefully timed relative to the previous beat in order to achieve a desired effect, may fall into the vulnerable period of the previous cardiac cycle and induce an arrhythmia. The risk of delivering a stimulation pulse during the vulnerable period increases if the recovery time changes from one beat to the next due to a change in activation pattern. The pattern of myocardial activation and recovery may change during premature ventricular contractions (PVCs) or other ectopic beats, accelerated beats, or other abnormal beats. It is undesirable to deliver extra systolic stimulation pulses immediately following such beats. A method for quickly and reliably classifying a cardiac beat is therefore needed to control the delivery of extra systolic stimulation pulses to follow normally conducted, non-pathologic, beats.

A cardiac beat includes the depolarization, or “activation”, phase of the myocardial cells, followed by the repolarization, or “recovery” phase. Ventricular depolarization is observed as the QRS complex on an ECG or intra-cardiac electrogram (EGM), and ventricular repolarization is observed as the T-wave. These signals represent the depolarization and repolarization of a mass of myocardial cells. The spatial pattern of activation, i.e., the pathway of a depolarization wavefront over the myocardium, will vary depending on where the beat has originated. Thus, the timing of activation and recovery relative to one another, known as the activation recovery interval (ARI) will change with changing patterns or origins of activation.

Normally, cardiac beats originate from the sinus node, the intrinsic pacemaker of the heart, and are referred to a “sinus beats.” However, as noted above, cardiac beats may be ectopic, such as premature atrial or ventricular contractions or re-entrant tachycardias or of other, non-sinus origin. The pattern of activation and recovery and ARIs can be measured at local sites by measuring the myocardial action potential. Marked changes in the spatial distribution of activation-recovery intervals measured from myocardial action potentials occur when the pattern of myocardial activation changes. Paced beats produce changes in activation-recovery intervals compared to intrinsic beats. Likewise, ectopic beats and re-entrant tachycardias or other non-sinus beats are expected to produce marked changes in activation-recovery intervals. -5

Measuring changes in activation patterns has been proposed for use in arrhythmia discrimination. In U.S. Pat. No. 5,257,621, issued to Bardy et al., an implantable cardioverter/defibrillator for detection and discrimination between tachycardia and fibrillation is generally disclosed including a method for identifying a predetermined fiducial point in the electrical signal associated with ventricular depolarization from each of two pairs of electrodes. The cumulative beat to beat variability of the intervals separating the two identified fiducial points is used to distinguish between ventricular tachycardia and fibrillation. A cardioelectric apparatus for the early detection of a tachycardia that generally includes means for measuring values for the heart rate and the action potential duration to derive a time-variant parameter to be compared to stored values to indicate if the derived parameter is in a tachycardia risk range is disclosed in U.S. Pat. No. 6,466,819 issued to Weiss.

Measuring changes in the Q-T interval has been proposed for use in atrial capture management. In U.S. Pat. No. 6,249,702, issued to van Oort, a method is generally disclosed for determining when a delivered atrial pace pulse has failed to capture the patient's atrium in a DDD or DDD(R) pacing system including measuring QT intervals and analyzing changes in QT intervals. When there has been a failure to capture the atrium, a ventricular pace is asynchronous with regard to the slower occurring natural atrial signal that comes after the failed atrial pace. The variation of successive QT interval values is greater in such situations than when atrial paces capture.

A need remains, however, for a method for quickly and reliably classifying cardiac beats that overcomes the various limitations described above with regard to rate or interval-based methods or other morphology-based methods. A cardiac beat classification method that may be performed as frequently as beat-to-beat, without requiring undue processing time, and is independent of rate and interval information may be used for improving the safety and performance of cardiac stimulation therapies by correctly and rapidly classifying individual cardiac beats and using such information in controlling the delivery of a therapy.

### SUMMARY OF THE INVENTION

The present invention provides a system and method for classifying cardiac beats based on activation-recovery intervals (ARIs) or an ARI-related parameter such as the spatial dispersion of activation, recovery or ARIs. The beat classification method may be used in monitoring and detecting cardiac rhythms and/or for controlling a cardiac stimulation therapy. The beat classification method includes acquiring a reference ARI for one or more known types of cardiac beats; measuring the activation-recovery interval of an unknown cardiac beat during cardiac activity monitoring; comparing the measured activation-recovery interval to the stored reference ARI(s); and classifying the cardiac beat based on the comparison between the measured ARI and the reference ARI(s).

The present invention may be practiced in an implantable or external cardiac monitoring device for use in detecting and classifying heart rhythms or in a cardiac stimulation device for use in controlling a stimulation therapy. An implantable device in which the present invention may be realized includes EGM sensing circuitry for receiving cardiac signals from a selected sensing electrode vector. The device further includes signal processing circuitry for: detecting an activation time according to a first fiducial point defined relative to the QRS-complex of a cardiac beat; detecting a recovery time according to a second fiducial point defined relative to the T-wave occurring in the same cardiac beat; and measuring the ARI as the interval occurring between the detected activation time and the consecutively detected recovery time.

The device further includes control circuitry for controlling device functions, which may be in the form of a microprocessor and associated memory for storing data related to device operations and beat classification data. Cardiac beat classification is accomplished through operations performed by processing circuitry and control circuitry for measuring and comparing ARIs of unknown cardiac beats to stored reference ARIs corresponding to known cardiac beats. When embodied as a cardiac stimulation device, the device further includes pulse generating output circuitry and output control circuitry, which operations thereof may be affected by beat classifications made based on ARI measurements. The device operates in conjunction with electrodes positioned in operative relation to the heart for sensing cardiac signals and for delivering cardiac stimulation pulses.

The spatial dispersion of activation time, recovery time, or ARI may alternatively be used to classify a beat. The spatial dispersion of activation, recovery, or ARI may be measured as the difference between detected activation times, recovery times, or ARIs, respectively, measured from two or more sensing vectors during the same cardiac cycle. Dispersion of activation, recovery or ARI may be measured during a known rhythm and stored as a reference dispersion measurement. During cardiac activity monitoring, dispersion may be measured on a beat-by-beat or less frequent basis and the dispersion measurement made on an unknown cardiac beat is compared to a reference dispersion measurement for classifying the cardiac beat.

In a cardiac stimulation device, ARI-derived beat classifications may be used for: classifying cardiac beats as capture or loss of capture beats for purposes of capture management; classifying beats as tachycardia beats or other arrhythmia beats for use in detecting or confirming the presence of an arrhythmia; classifying beats as sinus or non-sinus beats, of which non-sinus beats may include ectopic beats or arrhythmia beats which may be further discriminated according to ARI measurements, for use in controlling when a stimulation pulse or a stimulation therapy sequence is delivered.

In one embodiment, ARI-derived beat classifications are used to verify capture during single chamber, bi-chamber, multi-chamber or multi-site pacing. Reference ARIs or ARI ranges are stored during stimulation known to capture at each desired stimulation site and/or during stimulation known to result in loss of capture at each stimulation site, individually or in any combination. During pacing, the ARI is measured on a beat-by-beat or less frequent basis following pacing pulse delivery using one or more sensing electrode vectors. Measured ARIs are compared to one or more reference ARIs for classifying the paced beat as a capture or loss of capture beat.

In another embodiment, ARI-derived beat classifications are used to control the delivery of extra systolic stimulation. The ARI is measured during each beat -8 after which an extra systolic stimulation pulse is scheduled to be delivered. If the beat is classified as “abnormal”, which may be a classification as an ectopic beat, a tachycardia beat, or other type of beat not associated with normal sinus rhythm, the scheduled extra systolic stimulation pulse is withheld. ARIs may additionally or alternatively be measured during the extra systolic beat to classify the response as a normal response or an abnormal response, which classification may be used in controlling the delivery of future extra systolic stimulation pulses.

In yet another embodiment, ARI-derived beat classifications may be used for detecting or confirming an arrhythmia. An ARI measured for an unknown cardiac beat may be classified as a tachycardia beat when the measured ARI approximately equals a known tachycardia reference ARI. Tachycardia may be detected when a predetermined number of beats out of a given number of consecutive beats are classified as tachycardia beats based on ARI measurements alone or ARI measurement-related criteria used in combination with other detection criteria based on arrhythmia detection methods known in the art.

### BRIEF DESCRIPTION OF THE DRAWINGS

FIG. 1A is an illustration of an implantable cardiac stimulation device coupled to a patient's heart by three cardiac leads and in which the present invention may be usefully practiced.

FIG. 1B is an illustration of an alternative implantable cardiac stimulation device that includes subcutaneous ECG sensing electrodes.

FIG. 2 is a functional schematic diagram of the device shown in FIG. 1A.

FIG. 3 depicts a representative unipolar EGM signal illustrating one method for measuring activation time, recovery time, and the ARI.

FIG. 4 illustrates one method for measuring electrical dispersion using two representative unipolar EGM signals measured from two different sensing vectors during a selected cardiac cycle.

FIG. 5 is a flow diagram providing an overview of operations included in the present invention for classifying a cardiac beat based on ARI measurements.

FIG. 6 is a flow chart summarizing steps included in a method for controlling a cardiac pacing therapy according to beat classifications based on ARIs measured during stimulation.

FIG. 7 is a flow chart summarizing a method for classifying beats using measured ARIs for use in capture management during pacing therapies.

FIG. 8 is a flow chart summarizing steps included in one method for performing capture management during biventricular pacing using ARI-based beat classifications.

FIG. 9 is a flow chart depicting a method for classifying beats using ARI measurements for use in controlling extra systolic stimulation.

FIG. 10 is a flow chart summarizing steps for classifying beats following an extra systolic stimulation pulse for use in controlling extra systolic stimulation therapy.

FIG. 11 is a flow chart summarizing steps included in a method for detecting arrhythmias using ARI-based beat classification.

FIG. 12 is a flow chart summarizing steps included in a method for classifying cardiac beats based on ARI measurements which further includes steps for validating a change in ARI measurements that result in an abnormal beat classification.

### DETAILED DESCRIPTION OF THE INVENTION

As indicated above, the present invention is directed toward providing a method for use in an implantable system for classifying cardiac beats. The implantable system includes a set of electrodes, which may be located on one or more cardiac leads, for measuring EGM or subcutaneous ECG signals and an implantable medical device for receiving the signals and processing the signals to measure ARIs or the dispersion of activation, recovery or ARIs. The implantable device may be embodied as a monitoring device for receiving EGM and/or ECG signals and storing ARI or dispersion data and/or the resultant beat classifications. The implantable device may additionally be capable of delivering an electrical stimulation therapy. In such embodiments, the device is capable of classifying cardiac beats based on ARI or dispersion measurements alone or in combination with other methods known in the art, such as methods based on sensed events, event intervals, event rates, and/or EGM morphology. The device then controls the delivery of a stimulation therapy based on these beat classifications. Stimulation therapies may include, but are not limited to, cardiac resynchronization therapy, extra systolic stimulation therapies, bradycardia pacing, rate-suppression therapies, overdrive pacing therapies, anti-tachycardia pacing, and/or cardioversion and defibrillation therapies. Stimulation pulses or sequences of pulses administered during these therapies may be adjusted, withheld, or delivered based on beat classifications made according to ARI or dispersion measurements.

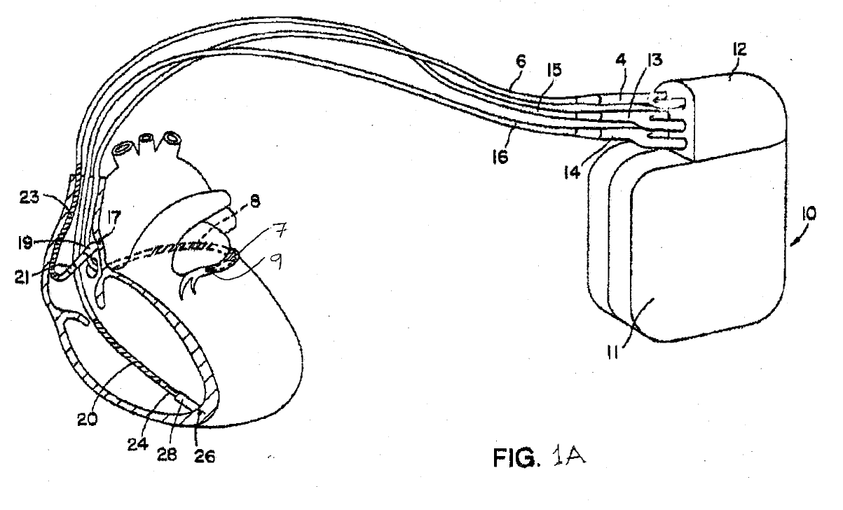

FIG. 1A is an illustration of an implantable cardiac stimulation device coupled to a patient's heart by three cardiac leads and in which the present invention may be usefully practiced. Device 10 is capable of receiving cardiac signals for monitoring purposes and delivering electrical pulses to achieve a therapeutic effect. Device 10 includes a connector block 12 for receiving the proximal end of a right ventricular lead 16, a right atrial lead 15 and a coronary sinus lead 6, used for positioning electrodes for sensing and stimulation in three or four heart chambers.

In FIG. 1A, the right ventricular lead 16 is positioned such that its distal end is in the right ventricle for sensing right ventricular cardiac signals and delivering stimulation pulses in the right ventricle. For these purposes, right ventricular lead 16 is equipped with a ring electrode 24, a tip electrode 26, optionally mounted retractably within an electrode head 28, and a coil electrode 20, each of which are connected to an insulated conductor within the body of lead 16. The proximal end of the insulated conductors are coupled to corresponding connectors carried by a lead connector 14 at the proximal end of lead 16 for providing electrical connection to device 10.

The right atrial lead 15 is positioned such that its distal end is in the vicinity of the right atrium. Lead 15 is equipped with a ring electrode 21 and a tip electrode 17, optionally mounted retractably within electrode head 19, for sensing and stimulating in the right atrium. Lead 15 is further equipped with a coil electrode 23 for delivering high-energy shock therapy. The ring electrode 21, the tip electrode 17 and the coil electrode 23 are each connected to an insulated conductor within the body of the right atrial lead 15. Each insulated conductor is coupled at its proximal end to a connector carried by a lead connector 13.

The coronary sinus lead 6 is advanced within the vasculature of the left side of the heart via the coronary sinus and great cardiac vein. The coronary sinus lead 6 is shown in the embodiment of FIG. 1A as having a coil electrode 8 that may be used in combination with either the coil electrode 20 or the coil electrode 23 for delivering electrical shocks for cardioversion and defibrillation therapies. Coronary sinus lead 6 is also equipped with a distal tip electrode 9 and ring electrode 7 for stimulating and sensing functions in the left chambers of the heart. The coil electrode 8, tip electrode 9 and ring electrode 7 are each coupled to insulated conductors within the body of lead 6, which provide connection to the proximal lead connector 4.

The electrodes 17 and 21, 24 and 26, and 7 and 9 may be used in sensing and stimulation as bipolar pairs, commonly referred to as a “tip-to-ring” configuration, or individually in a unipolar configuration with the device housing 11 serving as the indifferent electrode, commonly referred to as the “can” or “case” electrode. If device 10 is intended for delivering high-voltage cardioversion and defibrillation therapies, device housing 11 may also serve as a subcutaneous defibrillation electrode in combination with one or more of the coil electrodes 8, 20 or 23 for defibrillation of the atria or ventricles.

For the purposes of detecting activation and recovery times for measuring ARIs, bipolar “tip-to-ing” sensing vectors, unipolar tip-to-can sensing vectors, and vectors between any available tip or ring electrode to a coil electrode can be used to sense a local EGM signal. A “biventricular unipolar” sensing vector could be established between a tip or ring electrode located on right ventricular lead 16 and a tip or ring electrode located on coronary sinus lead 6 for sensing a relatively local EGM signal. Coil electrodes 8, 20 and 23 may be paired with the device housing 11 for sensing relatively more global EGM vectors for measuring more global activation and recovery times.

It is recognized that alternate lead systems may be substituted for the three lead system illustrated in FIG. 1A. For example, lead systems for sensing EGM vectors within a heart chamber may include one or more unipolar, bipolar leads and/or multipolar intracardiac and/or epicardial leads positioned in operative relation to one heart chamber. A single EGM sensing vector may be used for measuring ARIs for beat classification.

Alternatively, two or more ARIs may be measured from two or more EGM sensing vectors for beat classification. In some embodiments, ARIs may be measured in two or more heart chambers for classifying cardiac beats, particularly for use in classifying capture or loss of capture beats during bi-chamber or multi-chamber pacing. Lead systems for sensing EGM vectors within multiple heart chambers may include one or more unipolar, bipolar or multipolar leads positioned relative to the respective heart chambers. In summary, local and/or relatively more global EGM signals may be used for measuring ARIs from one or more sensing vectors in one or more heart chambers for use in classifying a cardiac beat. Measurement of the spatial dispersion of activation, recovery, and ARI for classifying cardiac beats, as will be further described below, requires at least two sensing vectors but may include multiple sensing vectors using electrodes positioned in operative relation to one or more heart chambers.

FIG. 1B is an illustration of an alternative implantable cardiac stimulation device that includes subcutaneous ECG sensing electrodes. Methods for classifying cardiac beats may be successfully employed in systems having subcutaneously or submuscularly placed electrodes, with or without intra- or epicardially placed electrodes. In FIG. 1B, housing 11 is provided with an insulative coating 35 with openings 30 and 32. The uninsulated openings 30 and 32 serve as subcutaneous electrodes for sensing relatively global subcutaneous ECG signals, which may be used, in accordance with the present invention, in measuring ARIs for beat classification. An implantable system having electrodes for subcutanteous measurement of an ECG is generally disclosed in commonly assigned U.S. Pat. No. 5,987,352 issued to Klein, incorporated herein by reference in its entirety. In alternative embodiments, multiple subcutaneous electrodes incorporated on the device housing 11 or positioned on subcutaneous leads extending from device 10 may be used to acquire multiple subcutaneous ECG sensing vectors for measurement of ARI dispersion. Multi-electrode ECG sensing in an implantable monitor is described in U.S. Pat. No. 5,313,953 issued to Yomtov, et al., incorporated herein by reference in its entirety.

While a particular multi-chamber cardiac stimulation device and lead system is illustrated in FIGS. 1A and 1B, methodologies for classifying cardiac beats and controlling stimulation therapies included in the present invention may be adapted for use with other single chamber, dual chamber, or multi-chamber implantable cardiac stimulation or monitoring devices.

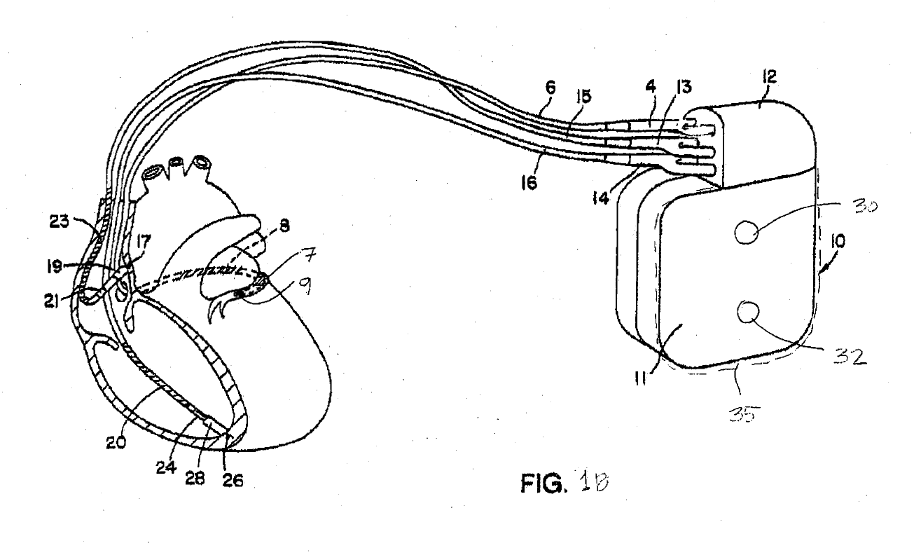

A functional schematic diagram of device 10 is shown in FIG. 2. This diagram should be taken as exemplary of the type of device in which the invention may be embodied and not as limiting. The disclosed embodiment shown in FIG. 2 is a microprocessor-controlled device, but the methods of the present invention may also be practiced in other types of devices such as those employing dedicated digital circuitry.

With regard to the electrode system illustrated in FIG. 1A, device 10 is provided with a number of connection terminals for achieving electrical connection to the leads 6, 15, and 16 and their respective electrodes. The connection terminal 311 provides electrical connection to the housing 11 for use as the indifferent electrode during unipolar stimulation or sensing. The connection terminals 320, 310, and 318 provide electrical connection to coil electrodes 20, 8 and 23 respectively. Each of these connection terminals 311, 320, 310, and 318 are coupled to the high voltage output circuit 234 to facilitate the delivery of high energy shocking pulses to the heart using one or more of the coil electrodes 8, 20, and 23 and optionally the housing 11.

The connection terminals 317 and 321 provide electrical connection to tip electrode 17 and the ring electrode 21 positioned in the right atrium. The connection terminals 317 and 321 are further coupled to an atrial sense amplifier 204 for sensing atrial signals such as P-waves. The connection terminals 326 and 324 provide electrical connection to tip electrode 26 and the ring electrode 24 positioned in the right ventricle. The connection terminals 307 and 309 provide electrical connection to tip electrode 9 and ring electrode 7 positioned in the coronary sinus. The connection terminals 326 and 324 are further coupled to a right ventricular (RV) sense amplifier 200, and connection terminals 307 and 309 are further coupled to a left ventricular (LV) sense amplifier 201 for sensing right and left ventricular signals, respectively.

The atrial sense amplifier 204 and the RV and LV sense amplifiers 200 and 201 preferably take the form of automatic gain controlled amplifiers with adjustable sensing thresholds. The general operation of RV and LV sense amplifiers 200 and 201 and atrial sense amplifier 204 may correspond to that disclosed in U.S. Pat. No. 5,117,824, by Keimel, et al., incorporated herein by reference in its entirety. Generally, whenever a signal received by atrial sense amplifier 204 exceeds an atrial sensing threshold, a signal is generated on output signal line 206. Whenever a signal received by RV sense amplifier 200 or LV sense amplifier 201 that exceeds an RV or LV sensing threshold, respectively, a signal is generated on the corresponding output signal line 202 or 203. Pacer timing and control 212 receives signals from signal lines 202, 203 and 206. Pacer timing and control 212 sets blanking intervals applied to sense amplifiers 204, 200 and 201 via A BLANK, RV BLANK and LV BLANK signal lines, respectively.

In one embodiment of the present invention, ventricular sense amplifiers 200 and 201 may include separate, dedicated sense amplifiers for sensing R-waves and T-waves, each using adjustable sensing thresholds. Activation times used for measuring ARIs may be detected when a signal exceeding an activation time sensing threshold is received by an R-wave sense amplifier included in RV or LV sense amplifiers 200 or 201, causing a corresponding activation time signal to be generated on signal line 202 or 203, respectively. Likewise, recovery times used for measuring ARIs may be detected when a signal exceeding a recovery time sensing threshold is received by a T-wave sense amplifier included in RV or LV sense amplifiers 200 or 201, causing a corresponding recovery time signal to be generated on signal line 202 or 203, respectively.

Pacer timing and control 212 may measure an RV ARI and an LV ARI as the time interval between an activation time signal and a consecutive recovery time signal received from RV and LV sense amplifiers 200 and 201, respectively. The dispersion of ARI may then be determined as the difference between the RV ARI and the LV ARI measured during the same cardiac cycle. The time interval between an activation time signal received from RV sense amplifier 200 and an activation time signal received from LV sense amplifier 201 during a given cardiac cycle may be measured as the dispersion of activation. Likewise, the time interval between a recovery time signal received from RV sense amplifier 200 and a recovery time signal received from LV sense amplifier 201 during a given cardiac cycle may be measured as the dispersion of recovery.

Switch matrix 208 is used to select which of the available electrodes are coupled to a wide band amplifier 210 for use in digital signal analysis. Selection of the electrodes is controlled by the microprocessor 224 via data/address bus 218. The selected electrode configuration may be varied as desired for the various sensing and stimulation functions of device 10. Signals from the electrodes selected for coupling to bandpass amplifier 210 are provided to multiplexer 220, and thereafter converted to multi-bit digital signals by A/D converter 222, for storage in random access memory 226 under control of direct memory access circuit 228.

Microprocessor 224 may employ digital signal analysis techniques to characterize the digitized signals stored in random access memory 226 to recognize and classify the patient's heart rhythm employing any of the numerous signal processing methodologies known in the art. In accordance with the present invention, digital signal analysis of selected EGM (or subcutaneous ECG signals if available) is performed by microprocessor 224 to measure activation and recovery times for measuring ARIs or electrical dispersion as will be described in greater detail below. In one embodiment of the present invention, any available electrodes may be selected in pairs by switch matrix 208 for use in determining activation and recovery times employing digital signal analysis methods applied to the selected EGM (or subcutaneous ECG) signal(s). Alternatively, circuitry for detecting myocardial recovery time for use in measuring ARIs may be provided as generally disclosed in U.S. Pat. Appl. No. XXXX (P11214) to Burnes et al., incorporated herein by reference in its entirety.

A telemetry circuit 330 receives downlink telemetry from and sends uplink telemetry to an external programmer, as is conventional in implantable medical devices, by means of an antenna 332. Data to be uplinked to the programmer and control signals for the telemetry circuit are provided by microprocessor 224 via address/data bus 218. Received telemetry is provided to microprocessor 224 via multiplexer 220. Numerous types of telemetry systems known for use in implantable devices may be used.

The remainder of the circuitry illustrated in FIG. 2 is an exemplary embodiment of circuitry dedicated to providing cardiac pacing, cardioversion and defibrillation therapies. Cardiac pacing therapies as used herein, refers to any cardiac stimulation therapy utilizing relatively low voltage, pacing class pulses, such as, but not limited to, bradycardia pacing, anti-tachycardia pacing, rate suppression pacing, rate overdrive pacing, cardiac resynchronization therapies, and extra systolic stimulation therapies. The pacer timing and control circuitry 212 includes programmable digital counters which control the basic time intervals associated with various single, dual or multi-chamber pacing therapies delivered in the atria or ventricles. Timing and control circuitry 212 also determines the amplitude of the cardiac pacing pulses under the control of microprocessor 224.

During pacing, escape interval counters within pacer timing and control circuitry 212 are typically reset upon sensing of RV R-waves, LV R-waves or atrial P-waves as indicated by signals on lines 202, 203 and 206, respectively. In accordance with the selected mode of stimulation, pulses are generated by atrial pacer output circuit 214, right ventricular pacer output circuit 216, and/or left ventricular pacer output circuit 215. The pacer output circuits 214, 215 and 216 are coupled to the desired electrodes for delivering stimulation pulses via switch matrix 208. The escape interval counters are also reset upon generation of pacing pulses, and thereby control the basic timing of cardiac pacing therapies.

If device 10 is configured to deliver cardiac resynchronization therapy (CRT), pacer timing and control 212 controls the delivery of cardiac pacing pulses at selected atrial-ventricular (A-V) and ventricular-ventricular (V-V) escape intervals, intended to improve heart chamber synchrony. Circuitry and methods for delivering a multi-chamber pacing therapy may be embodied, for example, as generally disclosed in U.S. Pat. No. 6,070,101 issued to Struble et al., or in U.S. Pat. No. 6,473,645 issued to Levine.

If device 10 is configured to deliver extra systolic stimulation (ESS) for the purposes of achieving post-extra systolic potentiation (PESP), pacer timing and control 212 controls the delivery of the ESS pulse according to an extra systolic interval. Circuitry and methods for delivering PESP stimulation may be embodied as generally disclosed in PCT Publication No. WO 02/053026 issued to Deno et al., incorporated herein by reference in its entirety, or the above-cited Bennett patent. The extra systolic interval may be controlled according to embodiments generally disclosed in the above-cited U.S. Pat. Appl. No. XXXX (P11214) to Burnes et al., or in U.S. Pat. Appl. No. XXXX (P11252) to Burnes, et al., also incorporated herein by reference in it entirety.

The durations of various timing intervals used by pacer timing and control 212 in delivering various pacing therapies, or cardioversion or defibrillation therapies, are set by microprocessor 224 via data/address bus 218. The microprocessor 224 includes associated ROM in which stored programs controlling the operation of the microprocessor 224 reside. A portion of the memory 226 may be configured as a number of recirculating buffers capable of holding a series of measured intervals for analysis by the microprocessor 224 for predicting or diagnosing an arrhythmia. In accordance with the present invention, memory buffers may be used to temporarily store measured ARIs and dispersions of activation, recovery, and ARIs for use in classifying a cardiac beat or in detecting, verifying or classifying an arrhythmia. The value of the count present in the escape interval counters when reset by sensed R-waves or P-waves can be also used to measure R-R intervals and P-P intervals for detecting the occurrence of a variety of arrhythmias according to rate and interval-based criteria.

In response to the detection of tachycardia, anti-tachycardia pacing therapy can be delivered by loading a regimen from microcontroller 224 into the pacer timing and control circuitry 212 according to the type of tachycardia detected. In the event that higher voltage cardioversion or defibrillation pulses are required, microprocessor 224 activates the cardioversion and defibrillation control circuitry 230 to initiate charging of the high voltage capacitors 246 and 248 via charging circuit 236 under the control of high voltage charging control line 240. The voltage on the high voltage capacitors is monitored via a voltage capacitor (VCAP) line 244, which is passed through the multiplexer 220. When the voltage reaches a predetermined value set by microprocessor 224, a logic signal is generated on the capacitor full (CF) line 254, terminating charging. The defibrillation or cardioversion pulse is delivered to the heart under the control of the pacer timing and control circuitry 212 by an output circuit 234 via a control bus 238. The output circuit 234 determines the electrodes used for delivering the cardioversion or defibrillation pulse and the pulse wave shape.

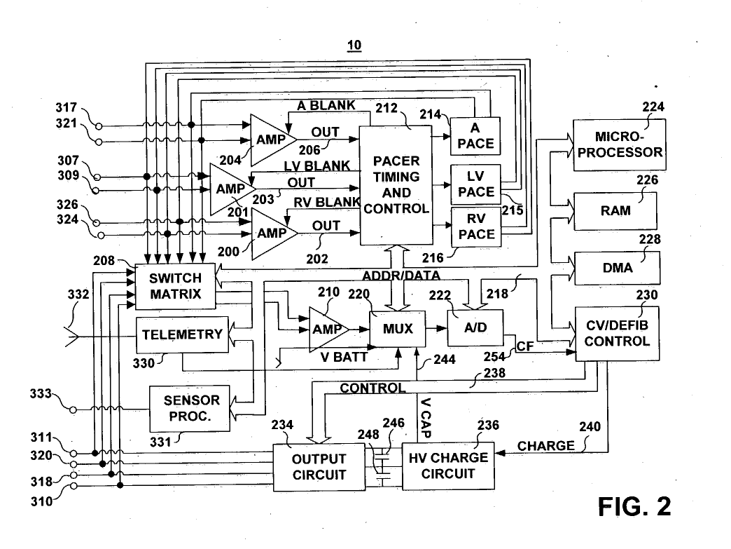

FIG. 3 depicts a representative unipolar EGM signal illustrating one method for measuring activation time, recovery time, and the ARI. ARIs may be measured from an EGM or subcutaneous ECG signal received from any available sensing electrode configurations as long as the resultant ARI varies a distinguishable amount dependently with changes in the pattern or origin of activation. The activation time is determined as the time a fiducial point defined relative to the QRS complex of a cardiac cycle is detected. The recovery time is determined as the time a second fiducial point defined relative to the T-wave of the same cardiac cycle is detected.

In the example of FIG. 3, a fiducial point for measuring activation time (AT) is selected as the maximum negative derivative of the QRS signal, dV/dtmin. The fiducial point for measuring recovery time (RT) is selected as the maximum positive derivative of the T-wave, dV/dtmax. The difference between the AT and RT is determined as the ARI. ARI measured as the interval on a unipolar EGM between the maximum negative derivative of the QRS signal and the maximum positive derivative of the T-wave has been shown to be closely correlated to the duration of the local monophasic action potential.

It is recognized that other fiducial points representing activation time and recovery time may be defined relative to the QRS complex and T-wave, respectively, for use in measuring an ARI. A fiducial point for measuring activation time may be selected as a minimum or maximum peak, a minimum or maximum derivative, a threshold crossing, a zero crossing or other identifiable characteristic of a QRS signal on a selected EGM or subcutaneous ECG sensing vector. The fiducial point used to detect activation time of an intrinsic depolarization may be different than, or the same as, the fiducial point used to detect activation time of an evoked depolarization. For example, if a pacing pulse has been delivered, the activation time may be determined as the time of pacing pulse delivery. A maximum or minimum peak, a maximum or minimum derivative, the end point of the T-wave, a threshold crossing, or other identifiable characteristic point on the T-wave signal may be selected as the fiducial point for measuring recovery time.

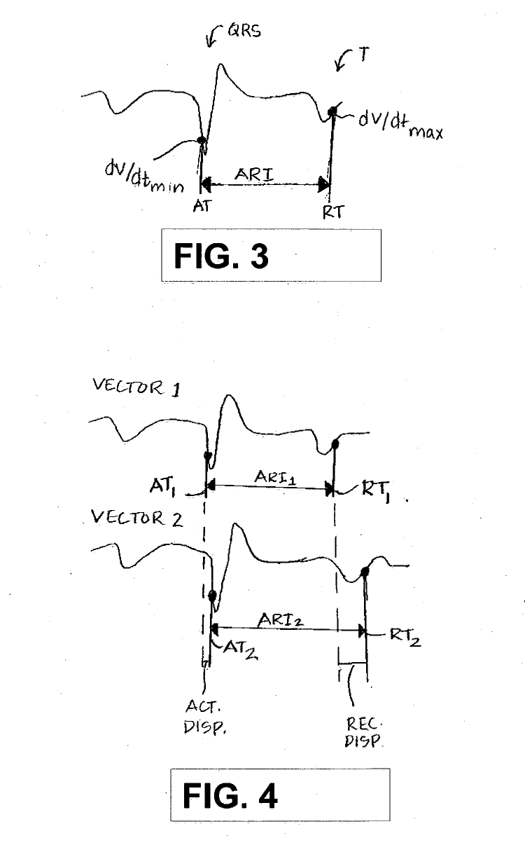

FIG. 4 illustrates one method for measuring electrical dispersion using two representative unipolar EGM signals measured from two different sensing vectors during a selected cardiac cycle. In some embodiments, a beat classification may be made based on one or more ARI measurements determined from one or more sensing vectors and/or the spatial dispersion of activation, recovery, or ARI. To measure spatial dispersion, the difference between the detected activation times, recovery times or measured ARIs from two or more sensing vectors is determined.

In FIG. 4, a first activation time, ATI₁; a first recovery time, RT₁; and a first activation-recovery interval, ARI₁, are measured from a first sensing vector (VECTOR 1) during a selected cardiac cycle. A second activation time, AT₂; a second recovery time, RT₂; and second activation-recovery interval, ARI₂, are measured from a second sensing vector (VECTOR 2) during the same selected cardiac cycle. The activation dispersion (ACT DISP) is the difference between the first and second activation times. The recovery dispersion (REC DISP) is the difference between the first and second recovery times. The ARI dispersion is the difference between the first and second ARIs, ARI₁ and ARI₂. Methods for measuring ARIs or the dispersion of activation, recovery and ARIs, which may be employed by the present invention, are generally disclosed in U.S. Pat. Appl. No. XXXX (P11215) to Burnes, et al., incorporated herein by reference in its entirety.

It is recognized that alternative methods for measuring or estimating an ARI or an ARI-related parameter (including the dispersion of activation, recovery, or ARI) may be substituted for the ARI and dispersion measurement methods described herein for use in classifying cardiac beats. For example, methods and circuitry for measuring Q-T intervals, such as disclosed in the above-cited patent to van Oort, may be adapted for use in the present invention by estimating an ARI based on Q-T intervals and classifying cardiac beats accordingly. Methods for measuring monophasic action potentials as generally disclosed in U.S. Pat. No. 6,152,882 issued to Prutchi; methods for estimating an action potential duration as generally disclosed in U.S. Pat. No. 6,522,904 issued to Mika et al.; or methods for determining a ventricular repolarization interval dispersion as generally disclosed in U.S. Pat. No. 6,456,880 issued to Park et al., all of which patents are incorporated herein by reference in their entireties, may be adapted for use in the present invention for obtaining an ARI-related parameter measurement for use in classifying a cardiac beat.

In the descriptions that follow, methods for classifying a cardiac beat generally refer to measuring an ARI. It is recognized that measurements of an ARI-related parameter, such as those discussed above, including measurements of the dispersion of activation, recovery or ARI, may be substituted wherever a measurement of ARI is referred to below for use in making a cardiac beat classification.

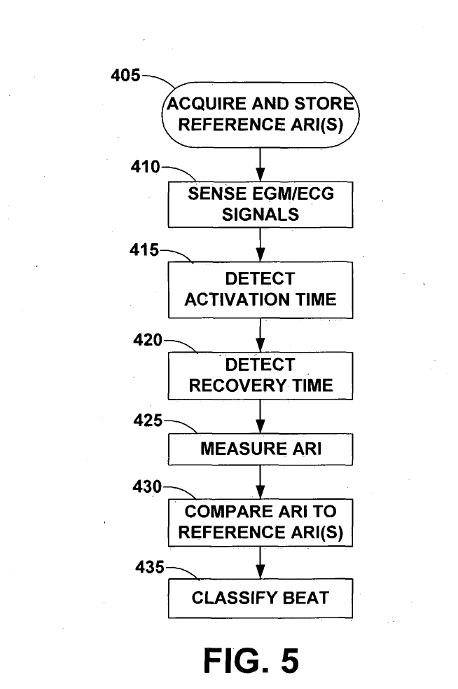

FIG. 5 is a flow diagram providing an overview of operations included in the present invention for classifying a cardiac beat based on ARI measurements. At step 405, reference ARIs that characterize one or more known types of cardiac beats are acquired. These one or more reference ARIs are stored in device memory to allow measured ARIs of unknown cardiac beats during cardiac activity monitoring or during a cardiac stimulation therapy to be compared to the reference ARIs for beat classification.

In one embodiment, a reference ARI is acquired by measuring the ARI during a desired number of cardiac cycles or interval of time using a selected sensing vector. The reference ARI may be determined as the mean or median value of a number of measured ARIs. A reference ARI may be defined as a range of intervals that may be set based on the range of ARIs measured over a given number of cardiac cycles or interval of time or as a defined percentage greater than or less than a mean or median ARI.

Reference ARIs are preferably acquired for normal sinus rhythm at one or more heart rates or heart rate zones. Since the ARI can vary with heart rate, reference ARIs corresponding to faster and slower rates may be useful in accurately discriminating normal, His-conducted, sinus beats from non-sinus beats. Reference ARIs can additionally or alternatively be acquired for other types of cardiac beats such as, but not limited to, premature ventricular contractions (PVCs), premature atrial contractions (PACs), beats associated with bundle branch block, tachycardia beats, supraventricular tachycardia beats, captured beats following a stimulation pulse, loss of capture beats following a stimulation pulse or fusion beats.

Reference ARIs may be updated periodically to account for changes in ARIs that may occur over time due to changes in electrode position, changes in physiological conditions, changes in medical therapies or other changes that may influence the ARI measurement. In some embodiments, reference ARIs may be updated automatically by storing a reference ARI as a running mean of a given number of consecutively measured ARIs.

Reference ARIs may alternatively be acquired and stored as user-entered values programmed into the implantable device according to expected ARIs for known types of cardiac beats or based on electrophysiological studies performed on an individual patient to evaluate changes in ARI during different cardiac rhythms or events. However, it is generally preferable that reference ARIs be based on intervals measured using the same sensing vector that will be used for monitoring cardiac activity by an implantable device since the ARI measured using one sensing electrode vector will generally be different than the ARI measured using a different sensing vector.

After storing a desired set of reference ARIs, cardiac activity monitoring may commence at step 410 by sensing an EGM or subcutaneous ECG signal from the selected sensing vector. At step 415, an activation time for a given cardiac cycle is detected according to a fiducial point on or relative to the QRS signal or a delivered stimulation pulse. At step 420, the recovery time is detected according to a fiducial point on or relative to the T-wave of the same cardiac cycle. The ARI for the cardiac cycle is measured at step 425 as the time difference between the detected activation time and consecutively detected recovery time.

At step 430, the ARI measured at step 425 is compared to one or more stored reference ARIs. By comparing a measured ARI during cardiac activity monitoring to a normal sinus reference ARI corresponding to the currently detected heart rate, the cardiac beat may be classified as “normal” if the measured ARI is approximately equal to the normal sinus reference ARI or within a normal sinus reference ARI range. If the measured ARI is substantially unequal to the sinus reference ARI, or outside the sinus reference ARI range, the beat may be classified as a non-sinus beat. This beat classification may be stored in a temporary buffer used for monitoring cardiac events or detecting arrhythmias. The beat classification may alternatively or additionally be stored in long-term memory to provide a log of cardiac beat classifications.

If the beat is not classified as a sinus beat, a measured ARI may be further compared to stored reference ARIs corresponding to PVCs or other ectopic beats, tachycardia beats, or other abnormal or arrhythmic beats. The beat may then be classified more specifically according to the reference ARI that most closely matches the measured ARI.

The beat classification method shown in FIG. 3 may be used alone for classifying a cardiac beat or in conjunction with other known beat classification methods. For example, a sensed cardiac event that is provisionally classified, for example as a PVC, tachycardia beat, supra-ventricular tachycardia beat, captured beat, loss of capture beat, fusion beat, retrograde-conducted beat, or other type of beat, based on rate, interval or other morphology-based methods may be verified as such by measuring the ARI of the subject beat and comparing the measured ARI to one or more reference ARIs. A beat classification made based on the measured ARI may thus be used to confirm or disprove a provisional beat classification by either matching or eliminating beat types based on reference ARI comparisons. Alternatively, ARI criteria may be included in a set of rules required to be met for making a beat classification along with other rate, interval or other morphology-related criteria.

ARIs may be measured for beat classification during cardiac activity monitoring for use in determining when a stimulation therapy is needed, such as an anti-arrhythmia therapy or an arrhythmia prevention therapy. ARIs may also be measured for beat classification during the delivery of a cardiac stimulation therapy for use in controlling the stimulation therapy. For example, the ARI may be measured following a stimulation pulse to determine if a normal, expected evoked response to the stimulation pulse has occurred. If the ARI is different than a normal evoked response ARI, the stimulation pulse may not have been effective, e.g. in a loss of capture situation, or the cardiac response to the pulse may be unfavorable due to changing physiological conditions, e.g. the genesis of an arrhythmia.

Thus, it is contemplated that ARI measurements for beat classification may be performed in conjunction with cardiac stimulation therapies which may include, but are not limited to, bradycardia pacing, cardiac resynchronization therapy, extra systolic stimulation, anti-tachycardia pacing, overdrive pacing, rate suppression pacing, and/or cardioversion or defibrillation therapies. If the ARI following a stimulation pulse is substantially different from the ARI expected of a normal evoked response to the pulse, the stimulation therapy may be adjusted or temporarily withheld.

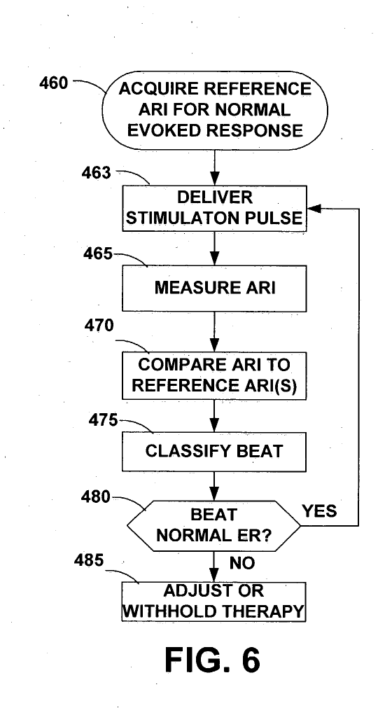

FIG. 6 is a flow chart summarizing steps included in a method for controlling a cardiac pacing therapy according to beat classifications based on ARIs measured during stimulation. At step 460, a reference ARI corresponding to a normal, evoked response to a delivered stimulation pulse is acquired and stored.

At step 463, a stimulation pulse is delivered according to the pacing therapy control parameters. The earliest activation time and consecutive recovery time -24 detected after the stimulation pulse is used to measure the ARI at step 465. The measured ARI is compared to the reference ARI corresponding to the normal expected evoked response to the stimulation pulse at step 470. The beat is then classified at step 475 as “normal” or “abnormal” according to the comparison made at step 470.

In one embodiment, capture verification may be performed by setting a recovery detection window centered approximately over the expected recovery time following a detected activation time (or delivered pacing pulse). The recovery detection window may extend, for example, 2 to 10 ms earlier and 2 to 10 ms later than the expected recovery time. If a recovery time is detected during the recovery time detection window, the beat is classified as a normal evoked response at step 475. If a recovery time is not detected during the recovery time detection window, the beat is classified as an abnormal beat at step 475.

If the beat is classified as a normal evoked response to the stimulation pulse as established by an ARI substantially equal to a normal evoked response reference ARI, as determined at decision step 480, the stimulation therapy continues to be administered without change by returning to step 463. If the beat is not classified as a normal evoked response to the stimulation pulse, as determined at decision step 480 and as established by a measured ARI substantially unequal to a normal evoked response reference ARI (or a recovery time falling outside a recovery time detection window) the pacing therapy may be withheld temporarily or adjusted depending on the beat classification made and the type of pacing therapy being delivered.

Additional diagnostic procedures known in the art may be performed at step 485 after detecting an abnormal beat, such as, but not limited to, analyzing the cardiac rhythm for detecting an arrhythmia or pro-arrhythmic state, diagnosing a lead-related problem, or verifying appropriate sensing thresholds. After a temporary withholding of therapy for one or more beats, or after adjusting the stimulation therapy, for example by changing stimulation electrodes, stimulation pulse energy, sensing threshold, or stimulation timing intervals, the therapy delivery may be restarted by returning to step 460, with continued monitoring of the cardiac activity during stimulation based on measured ARIs.

The method of FIG. 6 may be adapted according to the specific type of cardiac pacing therapy being delivered. One or more reference ARIs may be stored at step 460 relevant to the type of therapy being delivered, and the action taken at step 485 will depend on the type of therapy being delivered and the aspects of the therapy to be controlled. In one embodiment, as indicated above, beat classification is performed for the purposes of capture management.

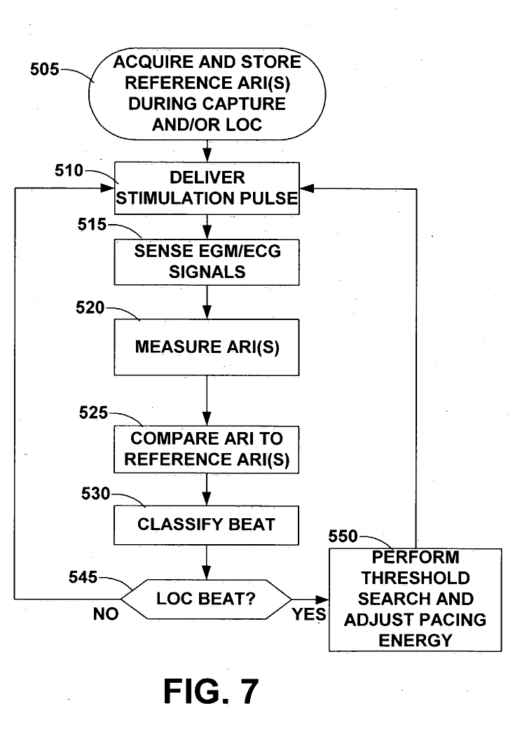

FIG. 7 is a flow chart summarizing a method for classifying beats using measured ARIs for use in capture management during pacing therapies. The method shown in FIG. 7 may generally be applied to single chamber, bi-chamber, multi-chamber, or multi-site pacing applications. At step 505, one or more reference ARIs are acquired and stored corresponding to capture and/or loss of capture beats during cardiac stimulation.

Cardiac activity monitoring commences during pacing by delivering a stimulation pulse at step 510 and sensing the EGM or subcutaneous ECG signals of one or more selected sensing vectors at step 515. At step 520, the ARI for each sensing vector is measured. The measured ARI(s) are compared to one or more reference ARIs at step 525. The beat is classified at step 530 as a captured beat or a loss of capture (LOC) beat based on the comparison made at step 525. If a LOC beat classification is made, as determined at decision step 545, a threshold search may be performed at step 550, and the stimulation pulse energy may be adjusted accordingly, before returning to step 520 to resume delivering the pacing therapy. If the beat is classified as a captured beat, no change is needed and the method returns to step 510 to continue delivering the stimulation therapy.

ARI-based beat classifications of capture and loss of capture beats may be used alone or in combination with other capture verification methods known in the art such as evoked response sensing or morphology analysis. A pacing threshold search may be performed according to known methods, for example those which generally rely on evoked response sensing following pacing pulses of varying pulse energy for identifying the lowest pulse energy at which an evoked response is detected. A threshold search may alternatively employ ARI-derived beat classification methods for identifying captured beats and non-captured beats during a pacing threshold test. The pacing threshold may be identified as the lowest pacing pulse energy at which the subsequently measured ARI is approximately equal to a known evoked response ARI or the pacing pulse energy at which the subsequently measured ARI is substantially different than the ARI measured for the next lower pacing pulse energy.

The method shown in FIG. 7 may advantageously be adapted for use in bichamber or multichamber capture management. For example, in the context of biventricular pacing for cardiac chamber resynchronization, the beat classification and pacing therapy control methods described in conjunction with FIGS. 6 and 7 may be adapted for biventricular capture management. As such, beats during biventricular pacing may be classified as biventricular capture, biventricular loss of capture, right ventricular capture with left ventricular loss of capture, or left ventricular capture with right ventricular loss of capture.

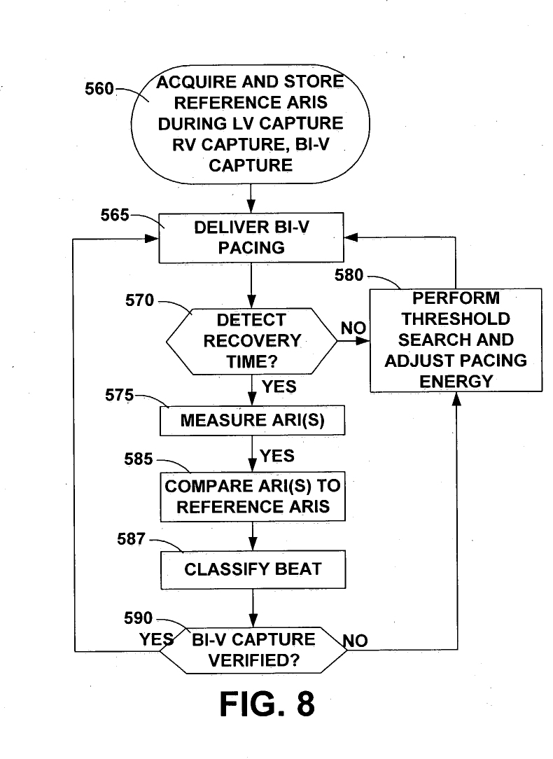

FIG. 8 is a flow chart summarizing steps included in one method for performing capture management during biventricular pacing using ARI-based beat classifications. At step 560, reference ARIs for are acquired and stored. Reference ARIs preferably include a reference ARI for left ventricular (LV) capture when the right ventricle is not captured; right ventricular (RV) capture when the left ventricle is not captured; and biventricular (BI-V) capture. Reference ARIS may be acquired from a single sensing vector or from an RV sensing vector and an LV sensing vector such that reference ARIs for LV capture and BI-V capture are acquired from the LV sensing vector and reference ARIs for RV capture and BI-V capture are acquired from the RV sensing vector. Alternatively or additionally, acquired and stored reference ARIs may correspond to loss of capture beats, i.e. biventricular loss of capture rather than biventricular capture. Any combination of sensing vectors and reference ARIs that allow for discrimination between capture and loss of capture in the right and left ventricles individually and simultaneously may be substituted for those described here. As described earlier, information about recovery times or activation times could also be used instead of or in combination with ARI. Furthermore, spatial or temporal dispersions of ARI, recovery, or activation times could be used for discriminating capture from loss of capture during bi-chamber or multi-chamber pacing.

Capture monitoring begins at step 565 with the delivery of biventricular pacing pulses. At decision step 570, a determination is made whether a recovery time is detected following delivery of the biventricular pacing pulses. Because the recovery time occurs later after the pacing pulse than the evoked R-wave, which is typically sensed for verifying capture, detection of the recovery time advantageously overcomes limitations associated with evoked response sensing due to post-pace polarization artifact.

A recovery time may be detected on one or more sensing vectors. If no recovery time is detected, loss of capture may have occurred in both chambers, and a pacing threshold search may be performed in both chambers at step 580. The pacing pulse energy may be adjusted according to the pacing threshold search results. Other diagnostics known in the art may be performed at step 580 to verify that a missed capture detection or true loss of capture is not related to a lead issue, inappropriate sensitivity settings, or other issue that may prevent accurate cardiac signal sensing.

If a recovery time is detected at decision step 570, the ARI is measured at step 575. If both an RV sensing vector and an LV sensing vector are being monitored, an ARI for each vector is determined from the respectively detected recovery times. An ARI may be determined as the interval between the delivered pacing pulse and the detected recovery time. In alternative embodiments, an activation time based on a fiducial point on the evoked QRS complex may be detected and used in measuring an ARI.

The measured ARI(s) are compared to the reference ARI(s) at step 585. This comparison is used to classify the beat at step 587. If a measured ARI interval is approximately equal to a reference ARI corresponding to biventricular capture (or substantially unequal to a reference ARI corresponding to biventricular loss of capture), the beat is classified as biventricular capture at step 587. Capture is verified at decision step 590, and the method of FIG. 8 returns to step 565 to continue biventricular pacing.

If a measured ARI interval is approximately equal to a reference ARI corresponding to RV-only capture or LV-only capture, i.e. one ventricle is captured and one ventricle is not captured, biventricular capture is not verified at step 590. The beat classification made at step 587 is one of RV capture with LV loss of capture or LV capture with RV loss of capture. A pacing threshold search is performed at step 580. The pacing threshold search may be performed in the chamber in which loss of capture is indicated according to the beat classification made at step 587 or optionally in both chambers.

If a measured ARI is substantially unequal to a reference ARI corresponding to biventricular capture or to RV-only or LV-only capture, (or is approximately equal to a reference ARI corresponding to biventricular loss of capture), the beat is classified as biventricular loss of capture at step 587. Since biventricular capture is not verified at decision step 590, a pacing threshold search may be performed at step 580. Other diagnostics known in the art for use in verifying or diagnosing a loss of capture detection may also be performed at step 580, e.g., to identify lead problems, make sensing threshold adjustments, etc. If the pacing threshold has changed in one or both ventricles, the pacing pulse energy is typically adjusted to a higher energy, greater than the new pacing threshold value, before returning to step 565 to continue biventricular pacing.

Measurement of the ARI for verifying capture is believed to be a more specific measure than evoked response sensing for capture management since an intrinsic R-wave (or other cardiac or non-cardiac signal) may occur at approximately the expected time of an evoked response, leading to false capture detections. By measuring the ARI, which directly reflects the activation and recovery pattern of the myocardium, evoked and intrinsic events can be specifically discriminated based on the difference in activation and recovery pattern associated with intrinsic and paced depolarizations.

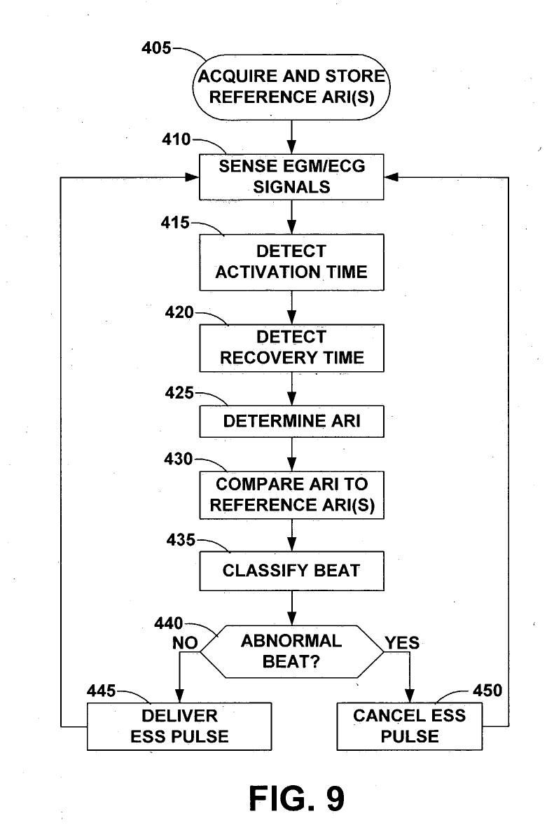

In other embodiments, ARI-based beat classifications may be applied to improve the safety of a cardiac pacing therapy. FIG. 9 is a flow chart depicting a method for classifying beats using ARI measurements for use in controlling extra systolic stimulation. It is generally undesirable to deliver an extra systolic stimulation pulse following a non-sinus beat. Therefore, ARI-based beat classification may be used on a beat-by-beat basis to discriminate sinus from other non-sinus beats to control the delivery of extra systolic stimulation pulses.

In FIG. 9, steps 405 through 435 correspond to identically numbered steps in FIG. 5, described above. A beat may be classified at step 435 as an abnormal or non-sinus type of cardiac beat because a measured ARI is either substantially unequal to a normal sinus reference ARI or substantially equal to a known, non-sinus cardiac beat reference ARI according to the comparison made at step 430. If an abnormal beat classification is made, as determined at decision step 440, a scheduled ESS pulse is canceled for that cardiac cycle at step 450. If the beat is classified as abnormal beat, which may be a PVC or tachycardia beat, it is undesirable to deliver an extra-systolic stimulation pulse due to the risk of inducing or accelerating an arrhythmia.

If the beat is classified as normal at step 435, as determined at decision step 440, the scheduled ESS pulse is delivered at step 445. After canceling or delivering a scheduled ESS pulse at step 450 or 445, respectively, the method shown in FIG. 9 may return to step 410 to sense the next cardiac event and measure the corresponding ARI.

Because the timing of an ESS pulse is critical to achieving post-extra systolic potentiation and avoiding stimulating during the vulnerable period, a change in the ARI, which directly reflects a change in the repolarization time of the myocardial cells, may result in an inappropriately timed ESS pulse. Therefore, a change in the ARI from the ARI associated with normal sinus beats may be used to detect abnormal beats during ESS therapy and thereby improve the safety of the therapy by controlling the delivery of ESS pulses to occur after normal beats and not after abnormal beats.

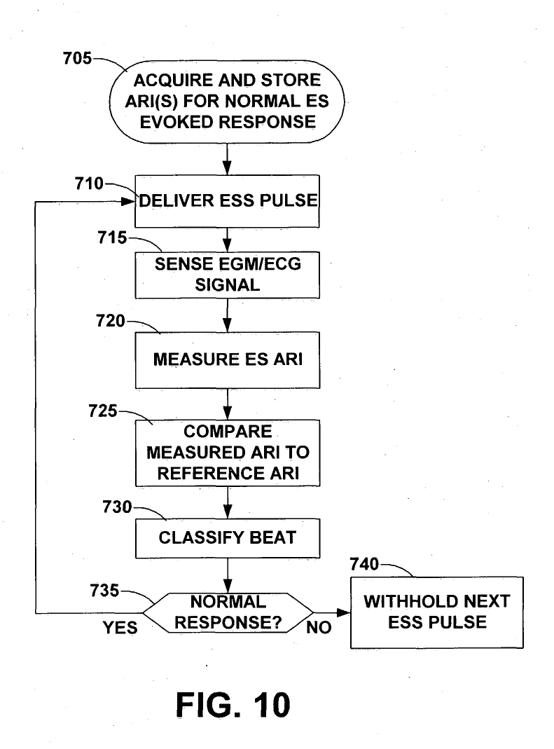

ARI-derived beat classifications may additionally be applied to the extra systolic beat to ensure that the expected response to the extra systolic stimulation pulse has occurred. As such, the method described previously in conjunction with FIG. 6 for classifying beats following a stimulation pulse may be applied for use during extra systolic stimulation therapy, as shown by the flow chart in FIG. 10. At step 705, a reference ARI corresponding to a normal extra systolic (ES) evoked response following an extra systolic stimulation pulse is acquired and stored.

At step 710, ESS begins with the delivery of an ESS pulse after which a selected EGM or subcutaneous ECG signal is sensed for the purposes of measuring the ES ARI at step 720. The ES ARI may be measured in the same manner as described previously for measuring normally evoked or intrinsic ARIs.

At step 725, the measured ARI is compared to the reference ES ARI, and at step 730 the ES beat is classified based on this comparison. If the measured ARI is approximately equal to the reference ES ARI, the ES beat is classified as a normal ES response, as determined at decision step 735, and the method of FIG. 10 may return to step 710 to continue delivering ESS. If the ES beat is classified as an abnormal ES response, as determined at step 735 and established by a measured ARI substantially unequal to the reference ES ARI, the next ESS pulse is withheld at step 740. A change in the ES ARI may reflect a change in myocardial refractoriness, potentially increasing the pro-arrhythmia risk during ESS. Therefore, ESS is preferably withheld for one or more cardiac cycles following the detection of an abnormal ES ARI, during which the cardiac rhythm may be monitored for arrhythmias based on ARI-derived beat classifications, rate or interval based criteria, other morphology-related criteria, or any combination of ARI-derived beat classifications and or other known arrhythmia detection methods.

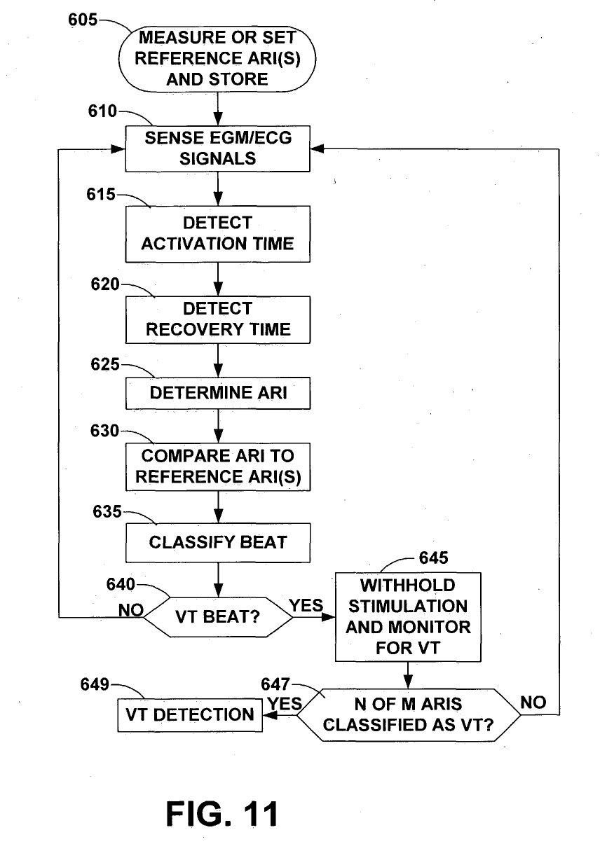

In some embodiments, ARI-derived beat classifications may be used for arrhythmia detection during cardiac rhythm monitoring for determining when an anti-arrhythmia or arrhythmia prevention therapy is needed. With regard to cardioversion and defibrillation stimulation therapies, beat classifications derived from ARI measurements may be used for detecting, or confirming a detection of, ventricular tachycardia or other arrhythmias, for determining when an anti-arrhythmia therapy is indicated. FIG. 11 is a flow chart summarizing steps included in a method for detecting arrhythmias using ARI-based beat classification. At step 605, reference ARIs are acquired and stored for known cardiac rhythms which may include, but are not limited to, sinus rhythm, ventricular tachycardia and supraventricular tachycardia.

Monitoring of cardiac activity commences at step 610 by sensing an EGM or subcutaneous ECG from one or more selected sensing vectors. The activation time and recovery time are detected during a cardiac cycle at step 615 and step 620, respectively, as described previously, for use in measuring the ARI at step 625.

By comparing the measured ARI to stored, reference ARIs at step 630, the current, unknown cardiac beat can be classified as a normal sinus beat, a pathological tachycardia beat, or other type of beat corresponding to a stored reference ARI. This beat classification may be made independent of event rate or interval information allowing arrhythmias to be detected even if the arrhythmia event rates or intervals are similar to event rates and intervals occurring during sinus rhythm.

If the beat is classified as a ventricular tachycardia (VT) beat, any pacing therapies that are being applied, such as cardiac resynchronization therapy, extra systolic stimulation, overdrive pacing, or rate suppression pacing, may be temporarily disabled at step 645 in order to eliminate blanking intervals applied to sensing circuitry during cardiac stimulation pulses. Such blanking intervals may be interfering with the ability of the implanted device to detect a high rate arrhythmia. Monitoring for VT or ventricular fibrillation may then continue at step 645 based on detecting a given number of VT beat classifications based on ARI measurements. If a predefined number, N, out of a given number of consecutive cardiac beats, M, are classified as Vr beats based on ARI measurements, as determined at decision step 647, a VT detection is made at step 649. If a VT detection criteria are not met at step 647, EGM/ECG monitoring continues by returning to step 610.

While the method shown in FIG. 11 specifies the detection of VT based on ARI-derived beat classifications, it is recognized that other forms of arrhythmias may be detected based on ARI-derived beat classifications. It is further recognized that other arrhythmia detection methods known in the art, e.g., rate, interval or other morphology based methods, may be used in combination with ARI-derived beat classifications for detecting arrhythmias. Classifying a cardiac beat based on ARI or ARI-related parameters may advantageously allow a pathological beat to be detected even when rate or interval-related detection criteria are not satisfied due to high rates being masked by sense amplifier blanking intervals or other forms of undersensing or due to pathological rates similar to normal sinus rates. Conversely, when rate or interval-related criteria for arrhythmia detection are met, classifying the cardiac beats based on ARI measurements allow verification of an arrhythmia detection based on changes in the activation pattern of the myocardium. By confirming or verifying a rate or interval-based arrhythmia detection using ARI-based beat classification, the incidence of false arrhythmia detections may be minimized when high sinus rates are present.

Factors may exist which cause a change in a measured ARI that is unrelated to a change in activation pattern or origin. Such factors may be lead-related, e.g. lead encapsulation, movement or dislodgement or compromised lead integrity. Therefore, in some embodiments, additional diagnostic methods or parameter monitoring may be performed as a cross-check for verifying that a change in a measured ARI is not due to factors other than changes in activation pattern. In addition or alternatively, the stability of an ARI measurement may be examined to determine if an ARI measurement reflects a transient change, such as in the presence of a premature ventricular contraction or non-sustained arrhythmia, or a sustained change, as might be expected when a lead-related factor has caused the ARI to change.

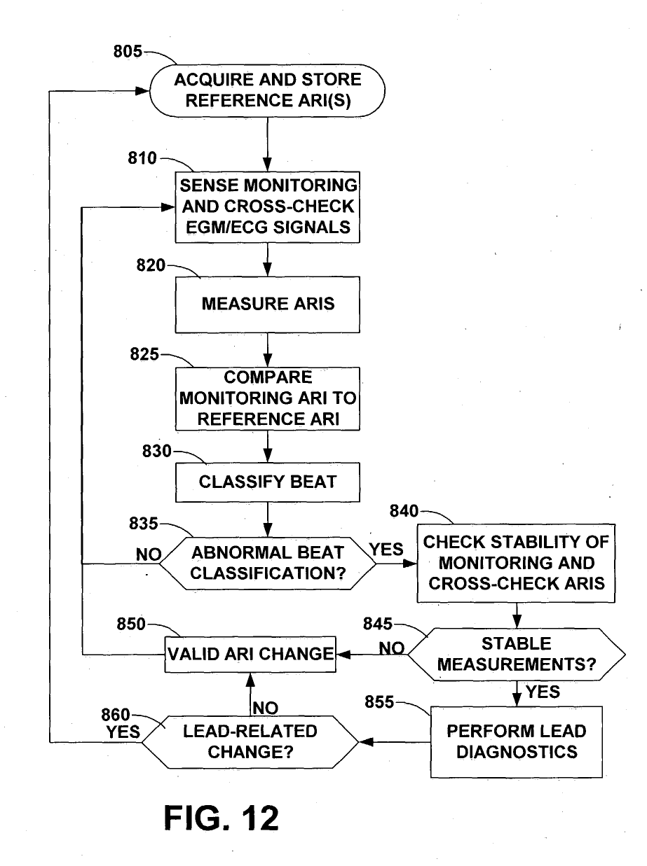

FIG. 12 is a flow chart summarizing steps included in a method for classifying cardiac beats based on ARI measurements which further includes steps for validating a change in ARI measurements that result in an abnormal beat classification. At step 805, reference ARI(s) are acquired and stored appropriate to the particular cardiac activity monitoring application. At step 810, an EGM or subcutaneous ECG signal selected for measuring ARIs for use in classifying cardiac beats, referred to as the “monitoring EGM signal,” is sensed. One or more additional EGM or subcutaneous ECG signal, referred to as a “cross-check EGM signal,” is sensed at step 810 for use in verifying that a change in ARI measured on the monitoring EGM signal is due to a change in activation pattern.

At step 825, ARIs are measured from both the monitoring and cross-check EGM signals. The ARI measured from the monitoring EGM signal is compared to the reference ARI(s) in order to classify the cardiac beat at step 830. If the beat is classified as a normal beat, as determined at decision step 835, cardiac activity monitoring continues by returning to step 810.

If an abnormal beat classification is made, as determined at decision step 835, the cross-check EGM signal may be used for verifying that a change in the activation pattern is present and the monitoring ARI measurement is valid. As such, at step 840, the stability of the monitoring and cross-check ARIs is examined to determine if the measurements have changed concurrently, reflecting an overall change in myocardial activation pattern, and if the change is a stable or instable change. If the monitoring and cross-check ARIs changed concurrently and if the change is instable or transient as determined at decision step 845, the ARI measurement is deemed valid at step 850. The concurrent changes in ARIs from two or more sensing vectors may indicate ectopy or a non-sustained arrhythmia. The method of FIG. 12 may return to step 810 to continue sensing the monitoring and cross-check EGM signals, measuring ARIs and classifying beats accordingly.

If, however, a change in the monitoring ARI measurement is not substantiated by a change in the cross-check ARI measurement, or the ARI measurements changes remain stable over a period of time without other evidence of a sustained arrhythmia, as determined at decision step 845, diagnostic tests for evaluating lead-related changes may be performed at step 855. Lead diagnostic tests may include lead impedance measurements or other lead performance tests known in the art. If a lead-related change is identified, as determined at decision step 860, new reference ARIs are preferably acquired by returning to step 805 prior to resuming cardiac activity monitoring. A change in lead impedance may indicate lead shifting or dislodgement, tissue encapsulation or other changes that influence the sensed EGM/ECG signals, thus influencing measured ARIs. The abnormal beat classification made at step 830 may be ignored for arrhythmia detection purposes.

If a lead-related change is not detected, as determined at decision step 860, the change in the monitoring ARI measurement is deemed valid at step 850, and cardiac activity monitoring may continue by returning to step 810. While the method shown in FIG. 12 relies on ARI stability measures or lead-related performance parameters for validating a measured change in ARI, it is recognized that, in alternative embodiments, other diagnostic procedures or cardiac activity parameters monitored by an implantable device for detecting abnormal beats or arrhythmias may be used as cross-checks for validating an abnormal ARI-derived beat classification.

While the illustrated embodiments depict implantable embodiments of the present invention those of skill in the art will recognize that external embodiments of the invention are well within the purview of the invention. For example, acute implementation of the present invention in an intensive care unit, emergency room or via an automated external defibrillator (AED) is expressly intended to be covered hereby.

Thus, a system and method for classifying cardiac beats based on ARI measurements have been described. The safety and performance of cardiac stimulation therapies may benefit from the use of ARI-based beat classifications by using the beat classifications to control therapy delivery, perform capture management, and improve the accuracy of cardiac rhythm monitoring methods.

1. A method for classifying cardiac beats, comprising:
storing a reference activation recovery interval-related parameter associated with a known non-pathological cardiac beat of a patient;
measuring an actual activation recovery interval-related parameter of an unknown cardiac beat;
comparing the measured parameter to the stored parameter; and
classifying the unknown cardiac beat according to the comparison made between the measured activation recovery interval-related parameter and the reference activation recovery interval-related parameter.

2. A method according to claim 1, wherein the measured parameter and the stored parameter comprise at least a one of: a spatial dispersion of activation time, a recovery time, an activation-recovery interval.

3. A method according to claim 1, wherein the classification relates to at least a one of: a loss of pacing capture event, a pacing capture event, a tachycardia event, a bradycardia event, a normal sinus event, a non-normal sinus event, a premature ventricular contraction event, a premature atrial contraction event, an ectopic focus depolarization event, a reentry circuit event.

4. A method according to claim 1, wherein the measuring step is triggered based on at least a one of: a predetermined part of a QRS ventricular depolarization complex, a portion of a T-wave, a portion of a P-wave, a detected activation event, a detected depolarization-recovery event.

5. A method according to claim 1, further comprising storing the classification of the unknown cardiac beat.

6. A method according to claim 1, further comprising storing the classification of the unknown cardiac beat on a beat-to-beat basis.

7. A method according to claim 1, further comprising storing the classification of the unknown cardiac beat in an aggregate classification-specific storage location with other cardiac beats having the same classification.

8. A method according to claim 1, wherein said measuring step is performed between a pair of electrodes, a first of said pair of electrodes comprising at least a one of: a distal tip electrode, a ring electrode, a coil electrode, a can electrode, a subcutaneous electrode, a surface electrode, a percutaneous electrode.

9. A method according to claim 8, wherein said first of said pair of electrodes is adapted to be disposed in communication with a one of: a ventricular chamber, an atrial chamber, a coronary sinus, a portion of a great vein, an epi-cardial location, an intrathoracic location, a transthoracic location, a superior vena cava location, a portion of a peripheral limb of a patient, a portion of a thorax of a patient.

10. A method for controlling a cardiac electrical stimulation therapy, comprising:
storing a reference activation recovery interval-related parameter associated with a known cardiac beat;
measuring an activation recovery interval-related parameter of an unknown cardiac beat;
comparing the measured parameter to the reference parameter;
classifying the unknown cardiac beat according to the comparison made between the measured activation recovery interval-related parameter and the reference activation recovery interval-related parameter; and
delivering, or withholding delivery of, a cardiac stimulation therapy based on the cardiac beat classification.

11. A method according to claim 10, wherein the cardiac stimulation therapy comprises at least a one of: a single-chamber therapy, a double-chamber therapy, a triple-chamber therapy, a quadruple-chamber therapy.

12. A method according to claim 10, wherein the cardiac stimulation therapy comprises at a one of the following pacing modalities: a DDD/R pacing modality, a DDI/R pacing modality, a VVI/R pacing modality, an AAI/R pacing modality, an ADI/R pacing modality, a DDD pacing modality, a DDD pacing modality, an AAI pacing modality, an ADI pacing modality, a VVI pacing modality.

13. A method according to claim 10, wherein the cardiac stimulation therapy comprises a bi-ventricular pacing modality.

14. A method according to claim 10, wherein the cardiac stimulation therapy comprises a bi-ventricular, cardiac resynchronization pacing modality.

15. A method according to claim 10, wherein the cardiac stimulation therapy comprises a dual chamber post extra-systolic potentiation pacing modality, wherein a extra-systolic stimulation pulse is delivered to a ventricle following a refractory period of said ventricle ends.

16. A method according to claim 15, wherein the measuring step is triggered based upon delivery of an extra-systolic stimulation pulse.

17. A method according to claim 10, wherein the measured parameter and the stored parameter comprise at least a one of: a spatial dispersion of activation time, a recovery time, an activation-recovery interval.

18. A method according to claim 10, wherein the classification relates to at least a one of: a loss of pacing capture event, a pacing capture event, a tachycardia event, a bradycardia event, a normal sinus event, a non-normal sinus event, a premature ventricular contraction event, a premature atrial event, an ectopic focus depolarization event, a reentry circuit event.

19. A method according to claim 10, wherein the measuring step is triggered based on at least a one of: a predetermined part of a QRS ventricular depolarization complex, a portion of a T-wave, a portion of a P-wave, a detected activation event, a detected depolarization-recovery event, delivery of a cardiac stimulation pulse.

20. A computer readable medium for storing executable instructions for controlling a processor to perform a method, said medium comprising:
instructions for storing a reference activation recovery interval-related parameter associated with a known cardiac beat;
instructions for measuring an activation recovery interval-related parameter of an unknown cardiac beat;
instructions for comparing the measured parameter to the reference parameter;
instructions for classifying the unknown cardiac beat according to the comparison made between the measured activation recovery interval-related parameter and the reference activation recovery interval-related parameter; and
delivering, or withholding delivery of, a cardiac stimulation therapy based on the cardiac beat classification.

21. A medium according to claim 20, wherein the cardiac stimulation therapy comprises at least a one of: a single-chamber therapy, a double-chamber therapy, a triple-chamber therapy, a quadruple-chamber therapy.

22. A medium according to claim 20, wherein the cardiac stimulation therapy comprises at a one of the following pacing modalities: a DDD/R pacing modality, a DDI/R pacing modality, a VVI/R pacing modality, an AAI/R pacing modality, an ADI/R pacing modality, a DDD pacing modality, a DDD pacing modality, an AAI pacing modality, an ADI pacing modality, a VVI pacing modality.

23. A medium according to claim 20, wherein the cardiac stimulation therapy comprises a bi-ventricular pacing modality.

24. A medium according to claim 20, wherein the cardiac stimulation therapy comprises a bi-ventricular, cardiac resynchronization pacing modality.

25. A medium according to claim 20, wherein the cardiac stimulation therapy comprises a dual chamber post extra-systolic potentiation pacing modality, wherein a extra-systolic stimulation pulse is delivered to a ventricle following a refractory period of said ventricle ends.

26. A medium according to claim 25, wherein the measuring step is triggered based upon delivery of an extra-systolic stimulation pulse.

27. A medium according to claim 20, wherein the measured parameter and the stored parameter comprise at least a one of: a spatial dispersion of activation time, a recovery time, an activation-recovery interval.

28. A medium according to claim 20, wherein the classification relates to at least a one of: a loss of pacing capture event, a pacing capture event, a tachycardia event, a bradycardia event, a normal sinus event, a non-normal sinus event, a premature ventricular contraction event, a premature atrial contraction event, an ectopic focus depolarization event, a reentry circuit event.

29. A medium according to claim 20, wherein the measuring step is triggered based on at least a one of: a predetermined part of a QRS ventricular depolarization complex, a portion of a T-wave, a portion of a P-wave, a detected activation event, a detected depolarization-recovery event, a delivered cardiac stimulation pulse.
